(19) United States
(12) Patent Application Publication
(10) Pub. No.: US 2020/0205789 A1
(43) Pub. Date: Jul. 2, 2020

(54) MEDICAL SAMPLE COLLECTION UNIT FOR PROCESSING AND ANALYSIS OF SURGICAL AND BODILY FLUIDS

(71) Applicants: Joshua D. Herwig, Memphis, TN (US); Esra Roan, Memphis, TN (US)

(72) Inventors: Joshua D. Herwig, Memphis, TN (US); Esra Roan, Memphis, TN (US)

(21) Appl. No.: 16/537,514

(22) Filed: Aug. 9, 2019

Related U.S. Application Data
(60) Provisional application No. 62/716,963, filed on Aug. 9, 2018.

(51) Int. Cl. A61B 10/00

(52) U.S. Cl. CPC A61B 10/0045

(57) ABSTRACT

A method and system of analyzing surgical and/or wound fluids from a live patient is provided. Such a system includes a medical diagnostic device, particularly one used in conjunction with wound drainage associated with general, plastic, oncologic, orthopedic, and neurological surgery utilized in conjunction with collected surgical and/or wound fluids. The ability to collect such drained surgical and/or wound fluids in a measured and uniform manner is undertaken in an effort to better understand the issues pertaining to such surgical and bodily fluids in terms of time and drainage rate to provide more effective analysis thereof than heretofore understood and undertaken.

### CROSS-REFERENCE TO CORRELATED APPLICATION

This application claims the benefit and priority to pending U.S. Provisional Application No. 62/716,963, filed Aug. 9, 2018, the entirety thereof the specification, drawings, and complete disclosures being incorporated herein by specific reference for all purposes.

### FIELD OF THE INVENTION

This invention relates to medical diagnostic devices, particularly those used in conjunction with analysis of bodily fluids and other related materials that may accumulate within the human body generally or in undesirable/erratic fashion. The ability to drain and collect such fluids and related materials (e.g. solids, films, and other like microorganism growths) from a person's (patient's) body, whether from a surgical or externally caused wound or buildup of fluids, etc. (such as a cyst or like structure), in a measured and standardized manner is undertaken within the disclosure in an effort to better understand the issues pertaining to such surgical/wound/buildup bodily fluids and materials in terms of time and drainage rate to provide more effective analysis thereof than heretofore understood. Such a system and method provide a novel approach to analyzing certain fluids/materials of this type and may permit a greater understanding as to the complexities of such human body internal concerns.

### BACKGROUND OF THE PRIOR ART

In order to drain the fluid which naturally builds up after surgeries such as hernia repair, abdominoplasties, mastectomies and lumpectomies, hip and knee surgeries, those used for the resolution of subdural hematoma, and the like, surgeons place drains attached to reservoirs which collect the bodily fluids for a period of time. However, even the most recent advances in the field, are unable to capture and store fluid in a robust and repeatable way, and therefore cause the fluid to be treated as waste only, and disallow or make difficult the study of the contents and characteristics of the fluid that is collected across all use cases. Fluid means a non-homogenous mixture or suspension comprised of, but not limited to wound exudate, serosanguinous fluid, blood, cells and cellular material, proteins, enzymes, cytokines, chemokines, inflammatory markers, growth factors, cellular debris including cytoplasmic fluid, cytosol, peptides, enzymes, and proteins or any byproducts which may form in the sample collection unit either naturally, or as a result of intentional modification of the sample or sample environment. etc. This is especially useful in the field of oncology for the determination of residual cancerous or tumorigenic cells and materials. Such fluids are subject to the current disclosure, as well as any materials related thereto.

In 2018, more than 1.7 million new cancer cases in the U.S. are projected by the American Cancer Society. For many, curative or palliative surgery remains the primary treatment with significant risk of recurrence. In pancreatic cancers, 5-year survival rate, after curative resection with no residual malignancy and clear margins, is 10-30%, attributed mostly to local recurrence. As such, recurrence or metastasis after curative surgery remains a real concern, which leads to delicate consideration of adjuvant treatment options before or after surgery. Local or systemic postoperative adjuvant therapies have considerable long or short term side effects. Therefore, it is desirable to identify patients that are at high-risk of recurrence for administration of post-operative therapies based on a variety of factors including disease stage and residual malignancy in the region. Post-operative fluid (seroma) produced in the surgical site has been considered for cytological or biochemical analysis for the detection of residual malignancy. However, seroma is produced during the patients' recovery at home and it is not possible or feasible to store, transport, and analyze.

Among the many factors, margin status, an indicator of residual malignancy, has been shown to correlate with decrease recurrence and re-excision rates. Margin status can be determined post- or intra-operatively. Although intra-operative techniques help in increasing margins, they are limited to providing information about malignancy only at the time of surgery. Margin status can also be detected by histopathologic analysis of the resected tissue postoperatively, but awaiting the results of the analysis may delay adjuvant therapy decisions. Collectively, existing methods inherently lack the capability to provide information about dormant malignant cells or cells that may be in the tissue surrounding the resection and in lymph nodes. As such, there is an increasing interest in the cytological analysis of post-operative fluid that is collected by closed suction drains or by fine needle aspirations in the days following a surgery. However, they remain in academic settings due to limitations of sample collection and downstream analysis as more and more patients now recover at home following oncologic surgery.

Fluid cytology has a significant role in detecting diseases of the lung and nervous system. Therefore, post-operative fluid (seroma) produced in the surgical site has been considered another potential specimen for cytological analysis storing information about residual malignancy. In a study of 142 mastectomy patients, 32 patients had seroma containing malignant cells postoperative day 6. Since this study in 1986, others have confirmed the presence of malignant cells in port-operative fluid in pancreatic cancer and breast cancer. The study by Ishikawa et al. showed that patients with benign tumors or non-invasive carcinomas had no malignant cells in seroma; and, mortality was higher with contaminated seromas. Most interestingly, by collecting fluid on 3 consecutive post-operative days, they showed that for 3 of the 94 patients in the study had negative results on first day shifted to positive results on day 2 and 3. This clearly highlights the importance of daily monitoring after surgeries. In breast surgery, malignant cells were not found in intraoperative washes, but in fluid collected with closed suction drains on post-operative day 2 even in those patients without axillary metastasis. Greenberg et al detected MUC-1 (a transmembrane protein overexpressed and aberrantly glycosylated in breast cancer cells) in 25% of the patients in axillary drainage collected on postoperative day 2. They found a correlation between MUC-1 presence and the number of metastatic lymph nodes. These studies provide strong evidence towards the use of post-operative fluid malignancy as marker of residual disease, which can aid recurrence risk assessment. However, no solutions exist to adequately provide for the repeated, measured, reliable, and convenient collection and analysis of the collected fluid. This is precisely the benefit of the present invention.

In addition to cytological detection, additional examinations of the seroma fluid were carried out for proteins such as the carcinoembryonic antigen (CAE) and MUC-1 with RT-PCR. In another study, exhausting analysis of chemokines, growth factors and cytokines was carried out with a commercially available array to indicate the differing profiles between benign and malignant sites.

Due to technological advances, flow cytometry has become a popular tool in identification of small tumors and circulating rare cells. Modern flow cytometers are able to process fluid samples 1mL/min rates where 10,000 cells/second may be analyzed. In addition, the detectible parameters have increased to double digits vs 1-2 in the past. As such, the use of flow cytometry to detect rare cells is popular, although with some limitations. In the case of detection of rare events, it is imperative to reduce processing and maintain the purity of the sample. In addition, it is useful to enhance the signal from the rare cells by finding labels specific for them. Flow cytometry is a robust, established method requiring minimal sample conditioning prior to analysis, and is thus a viable candidate technology for scaling of the device described herein.

It is clear that there is a strong interest in the analysis of the contents of the seroma fluid. Cytology is one way this can be achieved, but new advances in flow cytometry and other high throughput methods may provide interesting directions in the future. However, the collection of the fluid and maintaining its integrity for downstream analysis, while patients are mostly recovering at home away from healthcare facility is one problem that needs to be solved. Thus, the primary goal of this device is to enable the downstream analysis of seroma fluid in a patient-friendly manner for home use to unlock the information that is contained within seroma and to improve post-operative cancer care.

### ADVANTAGES AND SUMMARY OF THE DISCLOSURE

A distinct advantage of this disclosure is the ability to analyze surgical and/or wound fluids that have been collected in a controlled manner Another advantage of the disclosure is the beneficial diagnostic capabilities associated with controlled rates of surgical and/or wound fluid collection in terms of removal location, removal time period, and resultant type of surgical and/or wound fluid components.

Accordingly, this disclosure encompasses a system for analyzing collected bodily fluids and related materials [defined herein as fluids and solids, semi-solids, and/or film structures present within a surgical wound, externally caused wound, and/or an erratic internal accumulation (e.g., cyst, tumor, abscess, inflammation, and the like), as well as byproducts formed within such fluids and/or materials subsequent to such collection, whether naturally or in response to added materials prior to analysis] from a live patient, said system including at least one vacuum device attached to at least one fluid and material removal line, at least one collection component associated with both of said at least one vacuum device and said at least one fluid and material removal line, and at least one medical diagnostic instrument; wherein said at least one fluid and material removal line has an open end distal to said at least one vacuum device and configured for placement within or on a surgical wound; wherein said at least one vacuum device operates to transfer fluid from said wound to said at least collection component through said at least one fluid and material removal line; wherein said at least one collection component provides said collected surgical and/or wound fluid for introduction within said at least one medical diagnostic instrument; and wherein said at least one vacuum device provides a controlled rate of fluid and material removal from said live patient at specified times.

Such a system thus allows for a method of controlled collection of bodily fluids and related materials (as defined above) from a live patient coupled with the further utilization of such collected fluid for a concise analytical possibility of the components of such collected fluids. Such a method may thus encompass one of collecting bodily fluids and related materials from a live patient, said method including: a) the provision of at least one vacuum device attached to at least one fluid removal line and leading to at least one collection component associated with both of said at least one vacuum device and said at least one fluid removal line; b) placing said at least one fluid removal line on or within the body of said live patient (e.g. surgical wounds, surface or closed wound, thoracic space, and pleural space, without limitation); c) activating said at least one vacuum device at a controlled rate of fluid removal, thereby transferring surgical and/or wound fluid from said live patient to said at least one collection component; and d) discontinuing said collection component after a set period of time, leaving a resultant collection of removed fluid from said live patient; wherein said at least one collection component includes fluid analysis capabilities or enables/facilitates fluid analysis pertaining to at least one chemical or biochemical, physical or biophysical, optical, electromagnetic, or other relevant characteristic of the fluid or the constituents of the fluid. .

In other words, the ability to utilize a vacuum device with the other component parts of the system, one may provide not only a necessary removal of surgical wound, and/or internal accumulations of fluids and materials from a live patient (for protection from infection, at least), but further to do so in a controlled manner so as to permit a differentiated analytical procedure through collection in a manner that separates fluid components. Such separations may be through time measurements, density measurements (through, for instance, further centrifuging of such collected samples), pH meters, and other types of sensors (as noted in greater detail below). The controlled manner of collection thus opens up a myriad of possible ways of undertaking analytical processes in relation to such fluids, including, without limitation, the ability to determine initial component generation during wound healing (and thus in relation to actual collection times from a set starting point), rate of generation of certain fluid components, rates of immune responses within such healing wounds for a live patient, among many other considerations. Thus, the ability to control such surgical, wound and/or internal fluid and material collection processes through the utilization of such a system allows for analytical methods and diagnostic possibilities heretofore unexplored in the past, particularly as it concerns surgical and/or wound fluids. Thus, the collected bodily fluids and materials considered herein may be of any type of wound generated fluids (with or without solid, semi-solid, film-based, etc., materials present)(such as seromas, pus, growths, etc., without limitation), whether surgically based or created through an externally applied/introduced force and/or trauma, or naturally created by a subject patient's body in relation to an abnormal situation (again, such as a cyst, abscess, growth, fluid buildup, fluid/solid combination buildup, inflammation, etc., the list is myriad). The important consideration is the ability to collect such fluids and materials through a controlled rate removal device within predetermined time ranges in order to best assess fluid/material development, generation, mutation, modification, etc., within a specific time frame and at a specific collection rate for differentiated analytical potential, ultimately. Such a system and/or method thus accords, as noted throughout, the ability to analyze such fluids and/or materials in a manner that her heretofore been unexplored, particularly as it concerns such rates of collection. In the past, for instance, certain fluids and/or materials have certainly been biopsied for analytical purposes; however, such processes have merely concerned a cyst, tumor, etc., removal without any consideration as to the rate of collection thereof. Since, again, different results in actual structural, chemical, and/or biological composition of such bodily fluids and/materials over time during removal from a live patient, this disclosure provides a system and/or method that accords such beneficial possibilities unknown within the medical field in the past.

Additionally, the system and method may utilize a collection component that itself exhibits certain analytical components embedded, coated, and/or provided therein and/or thereon for assessment within the collection component itself, or as a manner of performing an initial analysis of such removed bodily fluids and materials prior to further introduction within a medical diagnostic instrument for further analysis. Furthermore, such a collection component may be structured as not only a collection chamber/bag/tube/etc., but also in terms of a structure that in and of itself facilitates such medical diagnostic instrument introduction subsequent to collection. Additionally, the system may include a filter component that removes certain bodily fluids and/or materials (particularly solids, semi-solids, and/or films) while the remaining collected fluids and/or materials pass to a different collection component, allowing then two different manners of collecting fluids and/or materials for analysis at the collection component site and/or for further introduction within a medical diagnostic instrument. The overall systems and methods associated therewith are described in greater detail below.

### BRIEF DESCRIPTION OF THE DRAWINGS

FIG. 1 is a schematic of one embodiment of the process of collection, shipment, analysis, and storage of the collected sample described in this document.

FIG. 2A is a view of one embodiment of the system used to facilitate collection of the sample, and the means to store the collected sample.

FIG. 2B is a view of one embodiment of the system used to facilitate collection of the sample and delivery of medically useful material to the patient, and the means to store the collected sample and medically useful material.

FIG. 3A is a view of several embodiments of the sample-collection apparatus in the downstream configuration with several embodiments of mechanisms to store data about the sample on the unit itself.

FIG. 3B is a view of several embodiments of the sample-collection apparatus in the downstream configuration shown with several embodiments of mechanisms to remove collected sample from the unit.

FIG. 3C is a view of two embodiments of the sample-collection apparatus in the downstream configuration shown with two embodiments of a sample-treatment substance contained in the apparatus.

FIG. 3D is a view of several embodiments of the sample-collection apparatus in the downstream configuration shown with two embodiments of a mechanism to remove sample from the apparatus, and one embodiment of a mechanism to process the sample without removing sample from the apparatus.

FIG. 3E is a view of one embodiment of the sample-collection apparatus in the downstream configuration shown with one embodiment of a mechanism to remove the sample from the apparatus.

FIG. 4A is a view of two embodiments of the sample-collection apparatus in the upstream configuration shown with two embodiments of a filter to collect the sample and a mechanism for transporting the sample.

FIG. 4B is a view of two embodiments of the sample-collection apparatus in the upstream configuration shown with two embodiments of sensors incorporated in the apparatus.

FIG. 5 is a schematic view of one embodiment of a mechanism for automatically removing, processing, analyzing, or processing and analyzing sample while in the sample-collection apparatus, and storing sample and sample data from the sample-collection apparatus in a large-scale operation such as a centralized or decentralized laboratory.

FIG. 6 is a schematic view of one embodiment of a mechanism for automatically removing, processing, analyzing, or processing and analyzing sample while in the sample-collection apparatus, and storing sample and sample data from the sample-collection apparatus in a small-scale operation such as home or hospital setting.

FIG. 7 is a schematic view of one embodiment of the overall process for collecting and shipping sample from the home to a centralized laboratory for sample processing, analyzing, and storing of the sample and sample data.

FIG. 8 is a schematic view of one embodiment of the overall process for collecting and shipping sample from the hospital or clinic to a centralized laboratory for sample processing, analyzing, and storing of the sample and sample data.

FIG. 9 is a schematic view of one embodiment of the overall process for collecting and shipping sample from a pharmacy to a centralized laboratory for sample processing, analyzing, and storing of the sample and sample data.

FIG. 10 is a schematic view of one embodiment of the overall process for collecting and shipping sample from the home to a decentralized laboratory for sample processing, analyzing, and storing of the sample and sample data.

FIG. 11 is a schematic view of one embodiment of the overall process for collecting and shipping sample from the hospital or clinic to a decentralized laboratory for sample processing, analyzing, and storing of the sample and sample data.

FIG. 12 is a schematic view of one embodiment of the overall process for collecting and shipping sample from a pharmacy to a decentralized laboratory for sample processing, analyzing, and storing of the sample and sample data.

FIG. 13 is a schematic view of one embodiment of the sample collection unit which possess on board sensors for analysis of the collected fluid, fluid constituents, or any byproducts which may form in the sample collection unit either naturally, or as a result of intentional modification of the sample or sample environment.

### DETAILED DESCRIPTION OF THE PREFERRED EMBODIMENTS AND DRAWINGS

In several embodiments, the present invention comprises an apparatus and method for the collection and rapid transport, processing, and analysis of collected surgical and bodily fluids (including wound exudate, serosanguinous fluid, or seroma fluid from the immediate vicinity of the surgical site following procedures relating to various forms of cancer including, breast, brain, liver, and pancreatic) for the near real-time information gathering and diagnoses of relevant disease states including cancer metastasis and tumorogenesis via the discovery of rare circulating tumorogenic or residual cancer cells in the perioperative space. Further characteristics which may be discovered include those elucidated by fluid cytology, and turbidity, proteins such as the carcinoembryonic antigen (CAE) and MUC-1, chemokines, growth factors and cytokines, cellular debris including cytoplasmic fluid, cytosol, peptides, enzymes, and proteins, gene profile, pH, cell count, presence of blood, presence of bacteria or other pathogens or infectious material or evidence of such, cell surface receptors or other markers for relevant disease states or conditions or any byproducts which may form in the sample collection unit either naturally, or as a result of intentional modification of the sample or sample environment. The timeliness, accuracy, and repeatability of the collected fluid is important in determining the progression of disease state or infection. The present invention provides a new, measured, repeatable, robust method of collecting, and facilitating the analysis of the previously described characteristics and improve post-operative surveillance of high-risk patients for recurrence and long-term outcomes. Furthermore, it allows for the instantaneous communication of such characteristic and analysis to the patient, caregiver, healthcare provider, or interested party. As such, any type of drainage instrument and/or device may be employed for such a purpose, particularly those that permit measured times and/or drainage rates for such surgical and/or bodily fluids removed from subject patient wounds. Thus, vacuum pumps, hydraulic pumps, fluid transfer devices, peristaltic pumps, and the like, may be utilized for such a purpose.

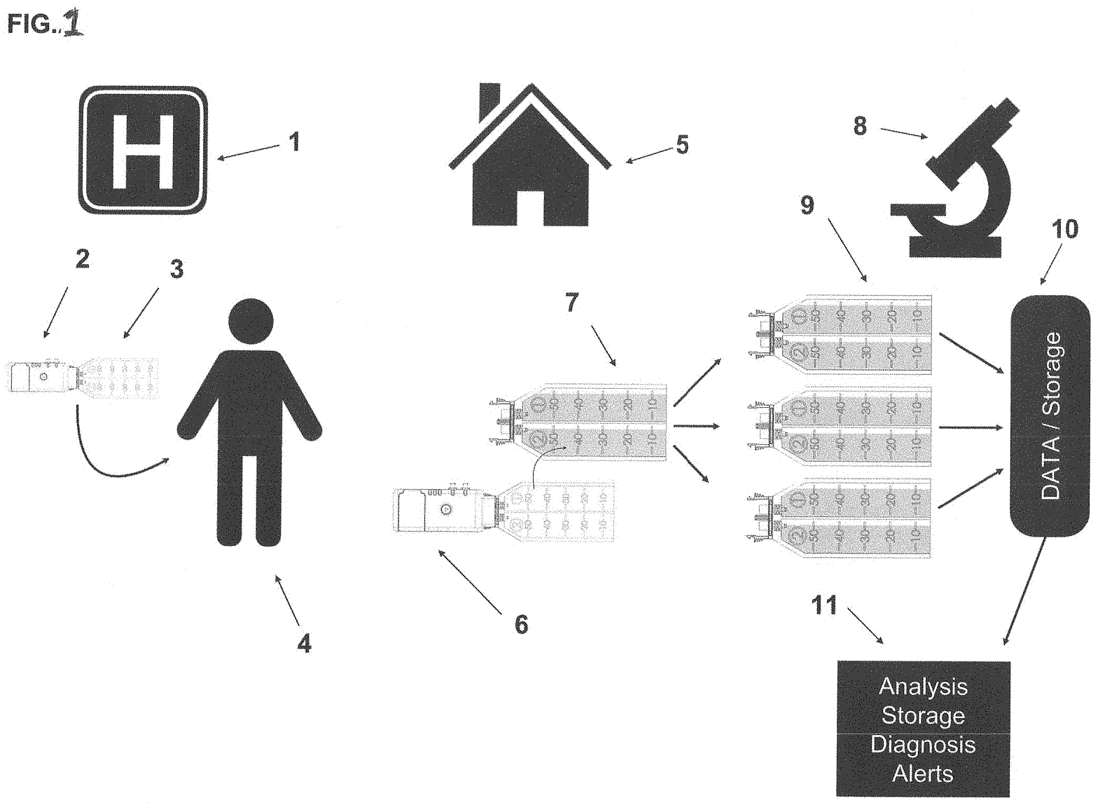

FIG. 1 shows an exemplary embodiment of the present system, and overview of the delivery of the system 2, 3 to the patient 4, normal operation of the system 6,7 in the home or hospital environment 5, and shipping and analysis of the collected bodily material (sample) 8, 9, 10, 11. The sample may be a bodily material belonging to one or more of the following: blood, urine, seroma, serosanguinous fluid, wound exudate from an open or closed wound including large flap-forming wounds, cells and cellular fragments, cellular debris, cytosol, peptides, enzymes, and other cytoplasmic fluid or constituents, proteins and protein-rich fluid, edema, cerebrospinal fluid, synovial fluid, or any other bodily fluid, tissue, or combination of the like.

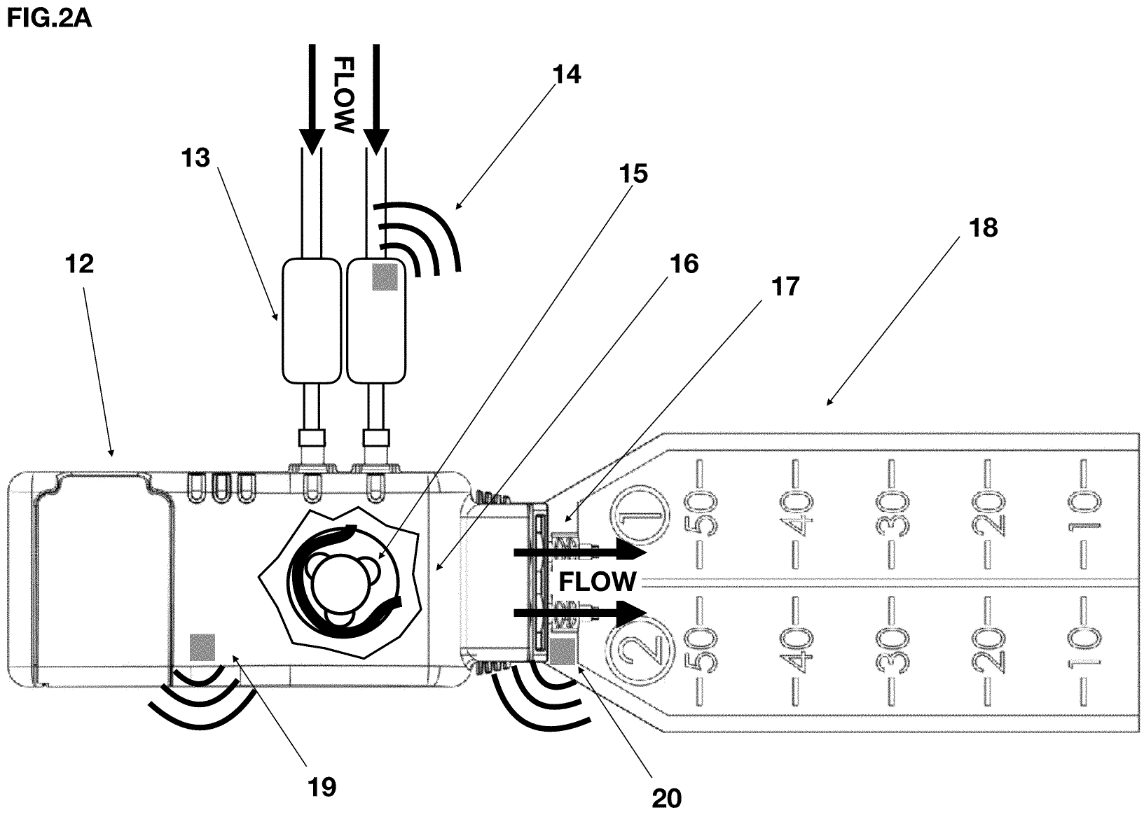

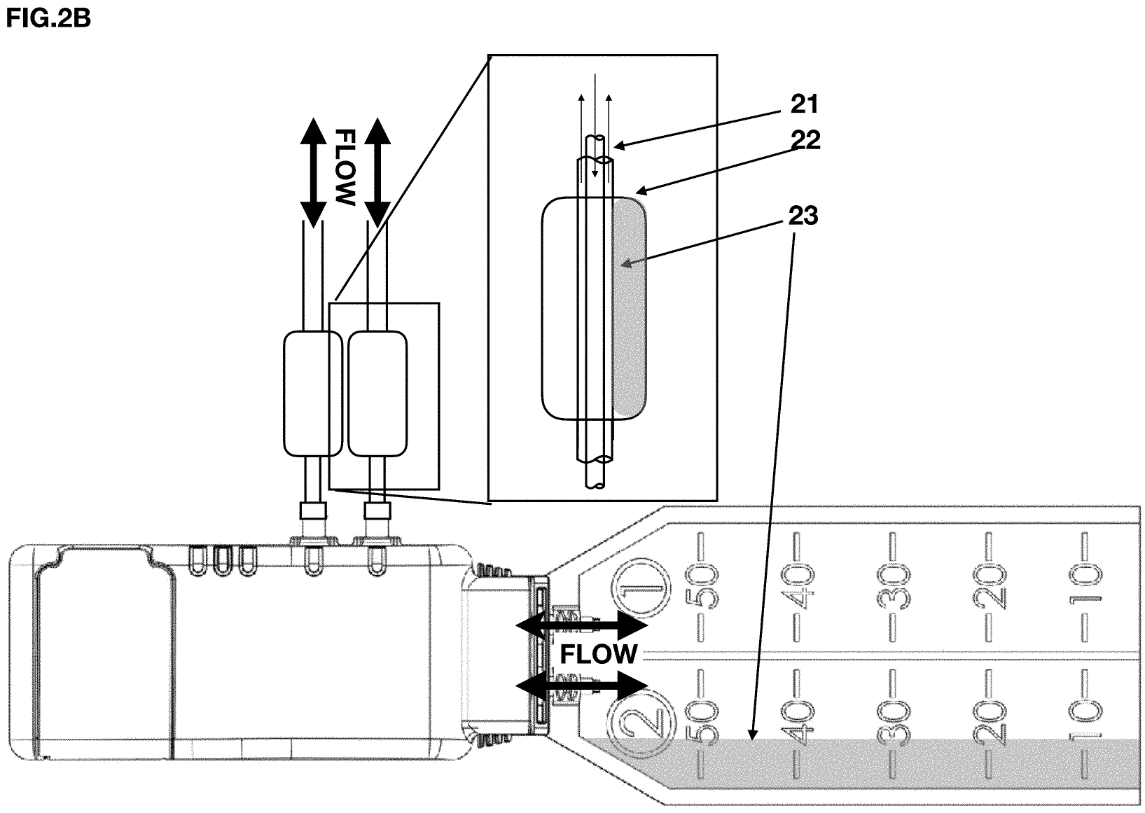

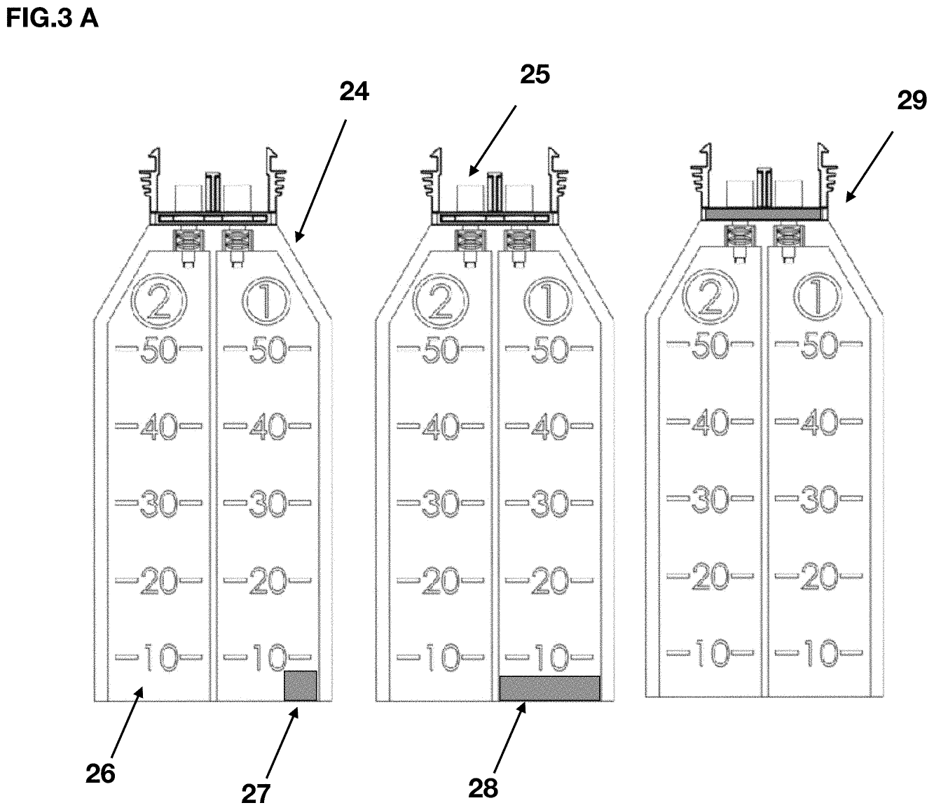

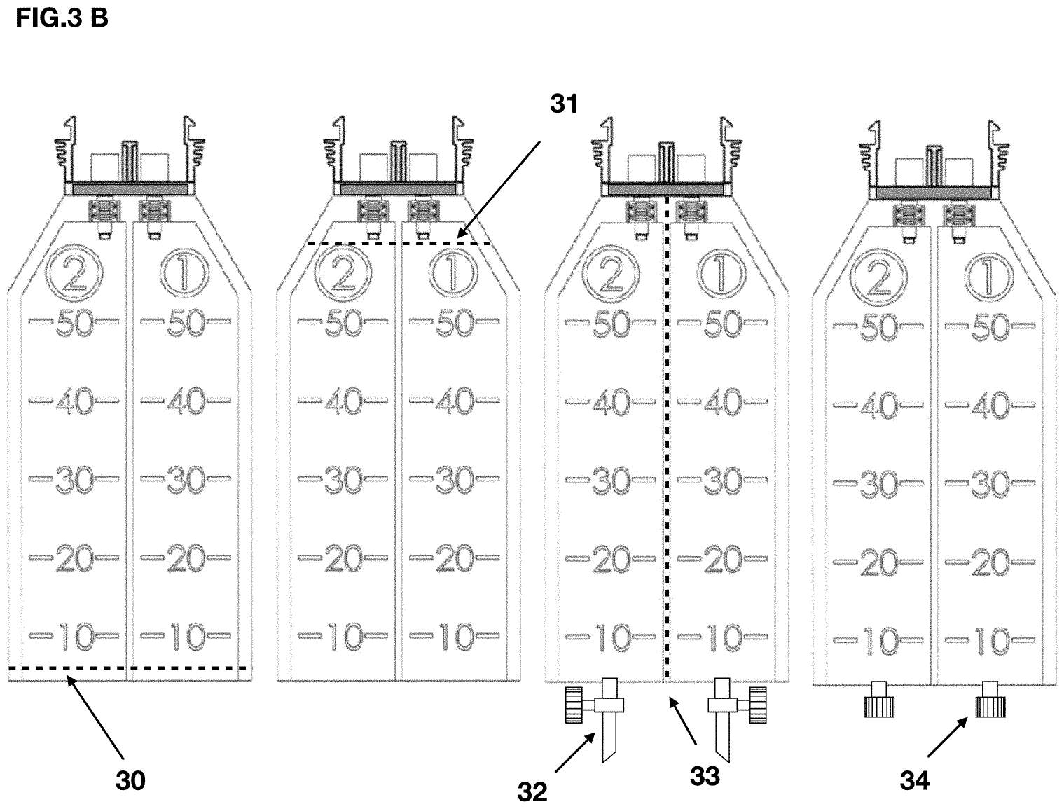

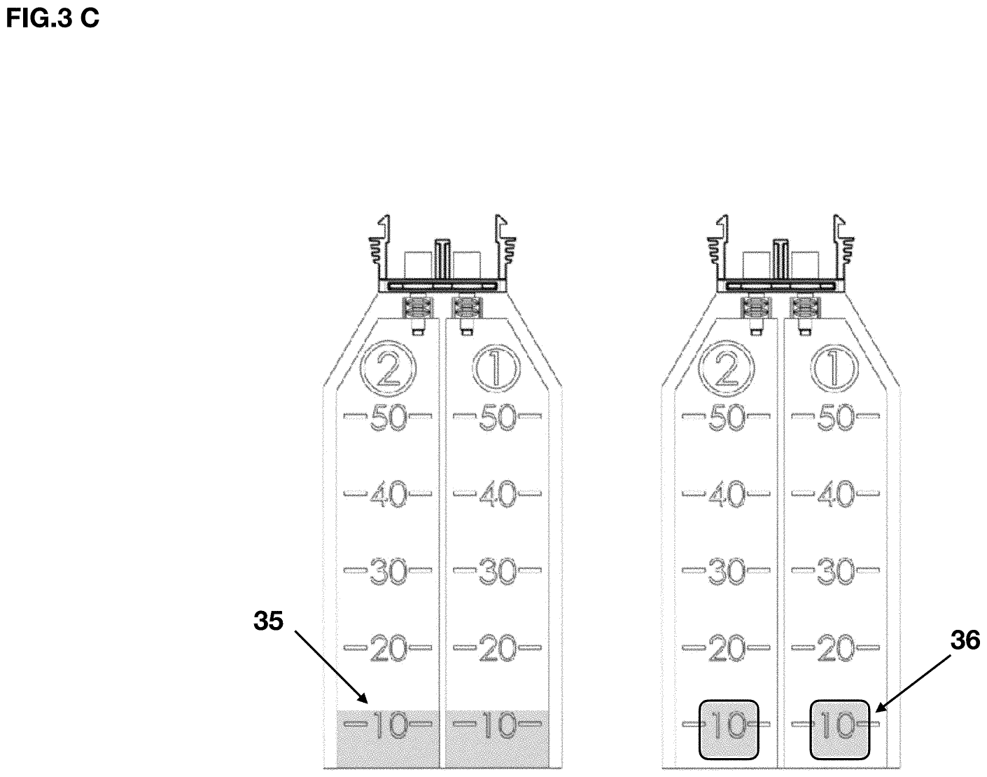

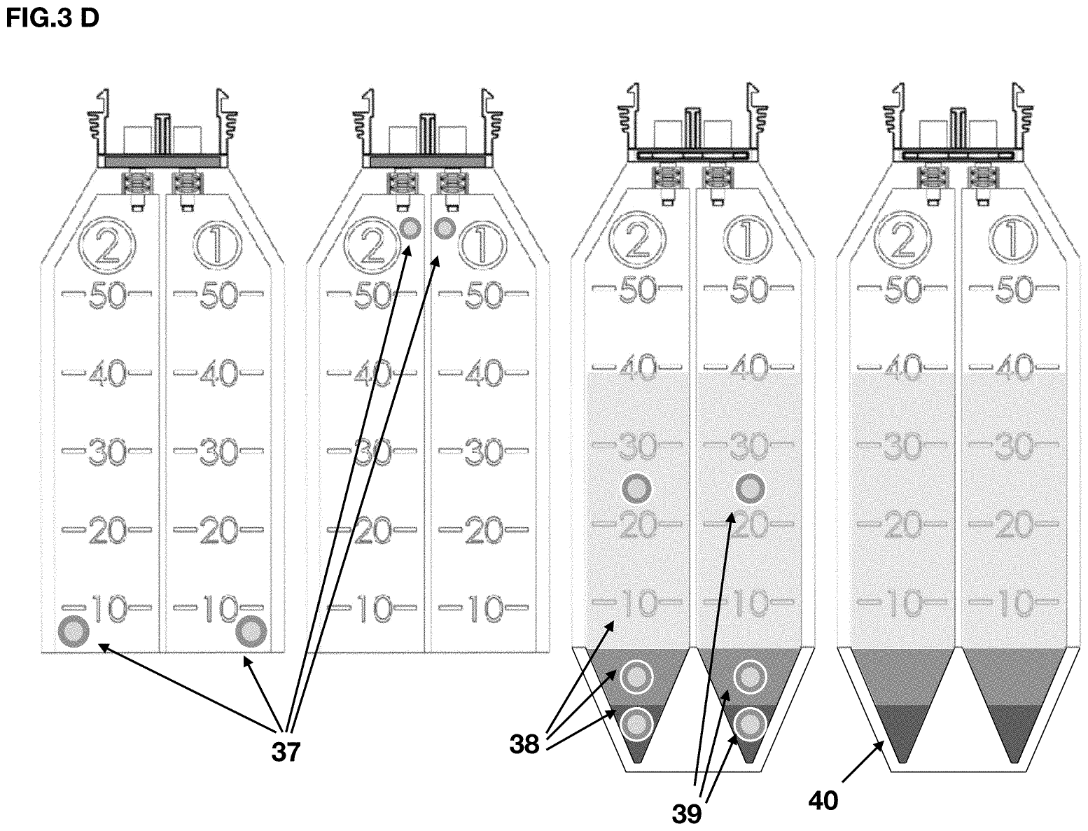

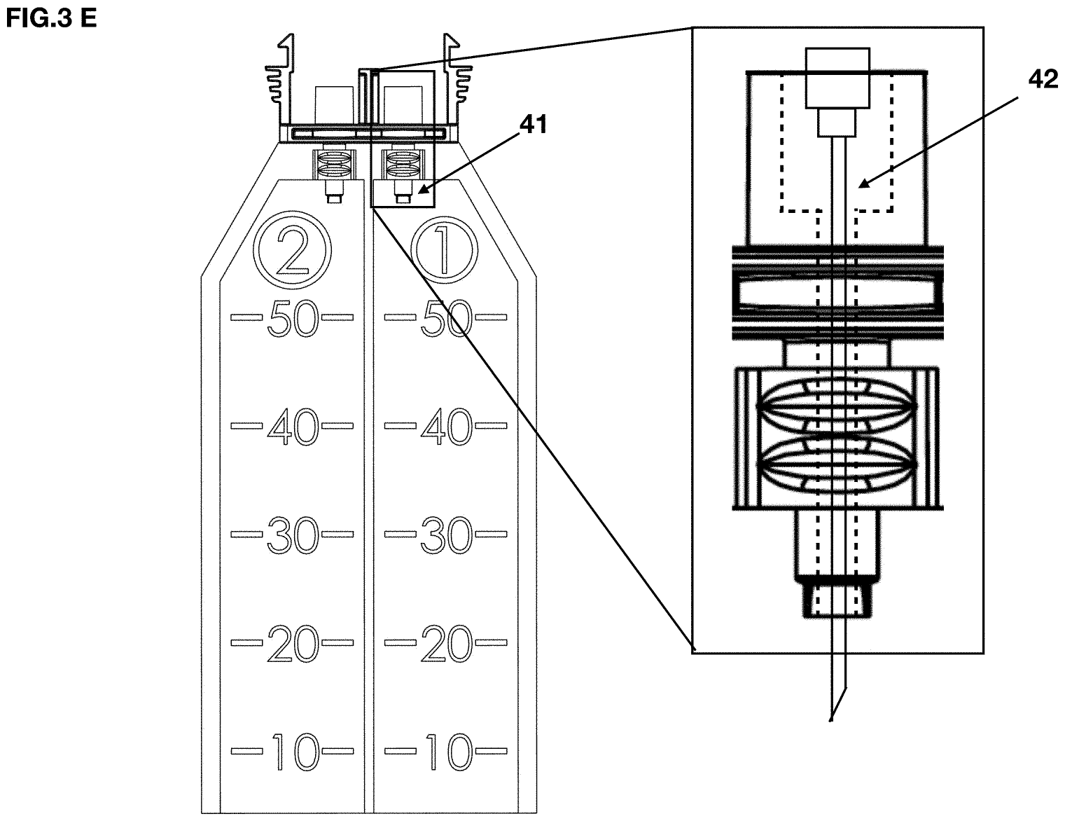

The sample-collecting apparatus 3 may be connected to a device 2, which enables the sampling of a relevant bodily material and transport of said material to the sample-collecting apparatus. The sample-collecting apparatus is further described in FIG. 3A-3E The device may do this by means of physical transfer via biopsy or the like, or through a liquid carrier such as serosanguinous fluid. The device may make use of negative pressure. The device is further described in FIG. 2A. In this embodiment, the device 2, which facilitates collection and transfer of the sample to the sample-containing apparatus 3 is placed onto the patient 4 in the hospital setting 1. The device may be installed on the patient to be continuously worn, or may be removable or transported not attached to the patient. The device may be pre-installed on the patient with one or more sample-containing apparatus 3, or may require attachment at a later time. The device may be installed on the patient 4 at the time of surgery or at any time during the patient's stay in the hospital, at home by a trained health-care provider or lay-person, or in any other appropriate setting including an inpatient or outpatient facility during a follow-up visit, a medical supply store, pharmacy, or the like. The device may be continuously used in the hospital or home setting 5, and may be transported on the patient, or with the patient during all other activities outside of the hospital or home setting. The device 6 and sample-containing apparatus 7, may continue to facilitate sampling of relevant bodily material over the time the device is used by the patient. The sample-collecting apparatus 7 may be removed from the device 6 and transported by some means including courier or other shipping services to a laboratory 8 or other facility for the purposes of analyzing some characteristic of the sample. These characteristics include, but are not limited to those elucidated by fluid cytology, and turbidity, the presence and characteristics of rare cells such as circulating tumor cells, proteins such as the carcinoembryonic antigen (CAE) and MUC-1, chemokines, growth factors and cytokines, cellular debris including cytoplasmic fluid, cytosol, peptides, enzymes, and proteins, gene profile, pH, cell count, presence of blood, presence of bacteria or other pathogens or infectious material or evidence of such, cell surface receptors or other markers for relevant disease states or conditions. Furthermore, the sample-collecting apparatus 9 may possess features that allow it to pre-process some or all of the desired analysis. In this embodiment, the sample, contained in the sample-collecting apparatus is probed by some means for data 10. This data is then stored, further probed or analyzed alone, or in combination with additional samples from the same or different patients. The relevant data or additional results may be transferred to the patient, their caregiver, health-care provider, or other interested party for diagnostic, prognostic, or research purposes. Additionally, the sample, itself may be stored, further probed or analyzed alone, or in combination with additional samples from the same or different patients. The sample may be transferred to the patient, caregiver, healthcare-professional, or other interested party for further analysis or storage.

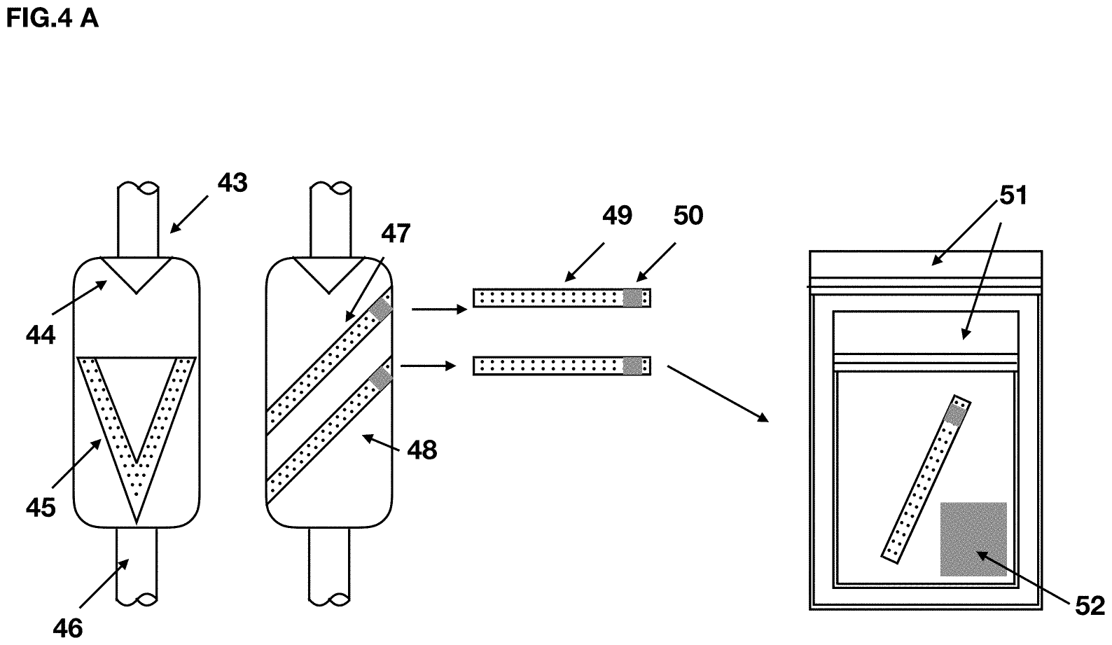

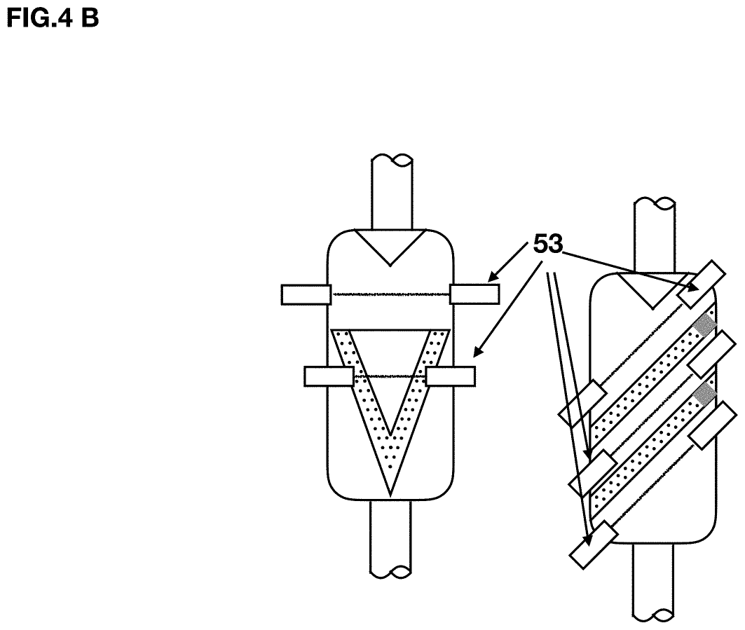

FIG. 2A-2B shows an exemplary embodiment of the device 12, described in the detailed description of FIG. 1 which may be necessary to facilitate collection and transfer of the sample described in the detailed description of FIG. 1 to the sample-collecting apparatus 17 described in the detailed description of FIG. 1. In this embodiment, the device is intended to create a negative pressure on the upstream (towards patient) side of a peristaltic mechanism 14, and a positive pressure downstream (away from patient) of the peristaltic mechanism (shown via a cutaway 16) which is intended to drive the collected sample into the sample-collecting apparatus. The device may possess multiple inlets. The sample-collecting apparatus may be connected to the device by a connection 16 and made to be detachable from the device and sent for additional testing as described in the detailed description of FIG. 1, or the entire apparatus and device may be transported for additional testing. In addition to a downstream sample-collection apparatus, an upstream sample-collection apparatus 13 may be connected upstream of the peristaltic mechanism. This upstream sample-collection apparatus is subject the same shipment protocol as described in the detailed description of FIG. 1. The device, upstream sample-collection apparatus, and downstream sample-collection may all possess features 14, 19, 20, which allow them to communicate with one another. In one embodiment, the upstream sample-collection apparatus and downstream sample-collection apparatus (separately or together) communicate with the device through some wired or wireless means, and the device communicates with the patient, caregiver, or health-care professional via wired or wireless means including making use of wireless cellular networks, satellite networks, Bluetooth or mesh network communication with a cellular device, modem, or other internet or communication-enabled device. The information communicated between the upstream and downstream sample-collection apparatus may include one or more of the following relevant characteristics of the collected sample: date of collection, elapsed time of collection, sample amount, sample chemical or biological characteristic, sample temperature at any or all times during collection, patient or sample identifying information, or shipping information. The device may then relay to the patient, caregiver, healthcare provider, or other interested party or process and encode back in the sample-collecting apparatus any or all of this information. The upstream sample-collection apparatus is further depicted in FIG. 4A-4B. The downstream sample-collection apparatus is further depicted in FIG. 3A-3E.

In one embodiment of the device, either the upstream or downstream sample-collection apparatus, or the device, itself, may contain a reservoir of material 23 intended to be delivered to the patient for the purposes of pain-relief, treatment of disease (including any form of cancer) or infection, or any other medically-useful purpose. This material may be delivered at one or more time-points based on several factors which are either pre-programmed, determined by the device or sample-collection unit based on patient or sample parameters determined by onboard analysis, or delivered to the device via some communication protocol or feature (e.g. the healthcare provider determines that the dosage of material should be increased, and is able to send instructions to the device to deliver material accordingly; the device may relay the patient or sample parameters to the healthcare provider for analysis in order for this determination to be made). The inlet to the device, which may include the upstream sample-collection apparatus may possess multiple cannulae 21, 22 which facilitate motion of fluid both out of, and into the patient. In one embodiment, sample is collected from the patient, analyzed by the device, and based on that analysis, material as herein described is transferred to the patient via the same or different cannula.

FIG. 3A-3E show an embodiment of the sample-collection unit or apparatus 24, into which fluid or biological sample is transferred after a collection process facilitated by a device as described in FIG. 2A-2B, or by the sample-collection apparatus itself. Furthermore, the sample-collection apparatus may serve as the terminal vessel in which all further analysis of its contents (i.e. the sample) is carried out, thus ensuring a secure, repeatable, non-contaminated sample during the chain-of-custody from collection to analysis results. The sample-collection apparatus and its associated packaging may be constructed to control, either actively or passively, characteristics of the sample and any associated packaging which include, but are not limited to the following: temperature, humidity, UV light transmittance and absorbance, shock and vibration, fluid ingress or egress, pH, gas ingress, egress, ambient concentration or absorbance, or any other biologically, clinically, or physically relevant characteristic. In one embodiment, the sample-collection apparatus is comprised of a flexible material with a specialized connector or inlet 25 which may facilitate easy installation and removal from the device as described in FIG. 2A-2B. The waste collection unit may be divided into one or more independent chambers, each of which are individually graduated with visible markings 26 to allow easy determination of the collected fluid. Furthermore, the sample-collection apparatus may possess some means of storing data pertaining to the collected fluid such as an Radio Frequency Identification (RFID) device 27 or barcode or quick response (QR) code 28, 29. The information contained in these devices is not limited to, but may contain any of the following: date of collection, elapsed time of collection, sample amount, sample chemical or biological characteristic, sample temperature at any or all times during collection, patient or sample identifying information, or shipping information.

In one embodiment, the sample-collection unit possesses features to facilitate removal of the collected sample. The sample-collection unit may possess perforations 30, 31 at either the top or bottom of the unit, which allow the unit to be easily opened. These perforations may be created in such a way as to not puncture the entirety of the collection unit, but rather form weakened points in the material, which facilitate origins for tearing or cutting the sample-collection unit. Furthermore the sample-collection unit may possess a stopcock 32, capped 34 or non-capped twist-open or squeeze-open outlet to allow easy removal of stored fluid. Additionally, the sample-collection unit may be easily divided into one or more separate collection and analysis chambers by means of a perforated seam 33, or other mechanism which facilitates separation.

In one embodiment, the sample-collection unit possesses a chemical or biologically-derived substance intended to preserve, store, or otherwise modify the sample or sample environment after sample collection. This substance 35 may be placed loosely in the sample-collection unit, or may be further contained in a pouch, bag, or capsule 36 intended to introduce the substance to the sample by either degradation, puncturing, bursting, or other method of the pouch, bag, or capsule. The substance may contain, without any limitation intended, one or more of the following: dilution of alcohol, pH buffer, protease inhibitor, anticoagulant (for blood, protein, or other substance), crosslinker, stain for imaging purposes, cell or DNA fixative, gene, protein, bacteria, or other marker for labeling via immunohistochemistry or other means.

In one embodiment, the sample-collection unit may possess a self-sealing syringe adapter or port 37 at various positions in the unit which facilitate easy removal of collected sample by syringe. Furthermore, the sample-collection unit may be constructed in such a way as to create a conical, or otherwise tapered section 40, in order to facilitate centrifugation of the sample in the sample-collection unit itself, thus avoiding transfer of the sample out of the unit to another vessel (such as a capped centrifuge tube). The centrifugation of the sample-collection unit may inherently create stratification of the sample constituents 38, which may be directly removed from the sample collection unit by syringe adapters or ports or other features which allows sample collection 39 placed at varying locations on the sample-collection unit. Furthermore, the tapered section may be created by a separable tube or vessel, which facilitates easy means of transfer from the sample-collection unit to the vessel; the separable vessel may allow easier handling and analysis of the sample, while maintaining the simplicity and security enabled by obviating the need to transfer the sample out of the sample-collection unit.

In a further embodiment of the sample-collection apparatus, the inlet to the apparatus may possess a feature 41 which allows one-way transfer of fluid during collection such as a one-way valve. It may further possess a channel 42 which is appropriately designed to allow the passage of a syringe needle through the inlet and one-way valve to facilitate sample removal from the unit. The removal via syringe or similar implement may be either manually actuated by the patient, a technician or trained person, or by automated means.

FIG. 4A-4B shows various embodiments of the sample collection unit in the upstream configuration, however all of the described features may also be implemented in the sample-collection unit in the downstream configuration. In one embodiment, the upstream sample-collection unit possesses an inlet 43, which may be connected to the patient by some means including commonly-used drainage tubing, or wound pad, and an outlet 46, which may be connected to the device as described in the detailed description of FIG. 2A-2B one-way valve 44 is placed immediately after the inlet which is designed to allow the passage of collected sample or fluid, but disallow the backflow of material (including air). The sample-collection unit may possess a filter, or several filters in either the conical configuration 45, or in a near-perpendicular-to-flow configuration 47, 48, which are selected based on filtration size, and are intended to capture various debris in the sample, for either removal, or collection for further analysis. The entire sample-collection unit, or one or more filters 49 may be individually or collectively shipped for further analysis. The filters or sample collection unit may possess features 50 which allow for the storage of data which may include but is not limited to date of collection, elapsed time of collection, sample amount, sample chemical or biological characteristic, sample temperature at any or all times during collection, patient or sample identifying information, or shipping information. The filters, or collection unit may be shipped in a single or doubled-bagged container 51, which itself may possess features 52 including barcodes, QR codes, or RFID tags which allow for the storage of data which may include but is not limited to date of collection, elapsed time of collection, sample amount, sample chemical or biological characteristic, sample temperature at any or all times during collection, patient or sample identifying information, or shipping information. The sample-collection unit, filters, or shipping bay may also be capable of communicating with the device (as described in the detailed description of FIG. 2A-2B), patient, caregiver, healthcare provider, or interested party.

In one embodiment of the sample-collection unit, sensors 53 may be used to detect parameters including, but not limited to those elucidated by fluid cytology, and turbidity, the presence and characteristics of rare cells such as circulating tumor cells, proteins such as the carcinoembryonic antigen (CAE) and MUC-1, chemokines, growth factors and cytokines, cellular debris including cytoplasmic fluid, cytosol, peptides, enzymes, and proteins, gene profile, pH, cell count, presence of blood, presence of bacteria or other pathogens or infectious material or evidence of such, cell surface receptors or other markers for relevant disease states or conditions. Furthermore, sensors may be used to determine with the filter or sample-collection apparatus has reached capacity for the material it is intended to collect. The data collected by these sensors may be stored in implements in the sample-collection apparatus, filter, or transferred to the device described in the detailed description of FIG. 2A-2B.

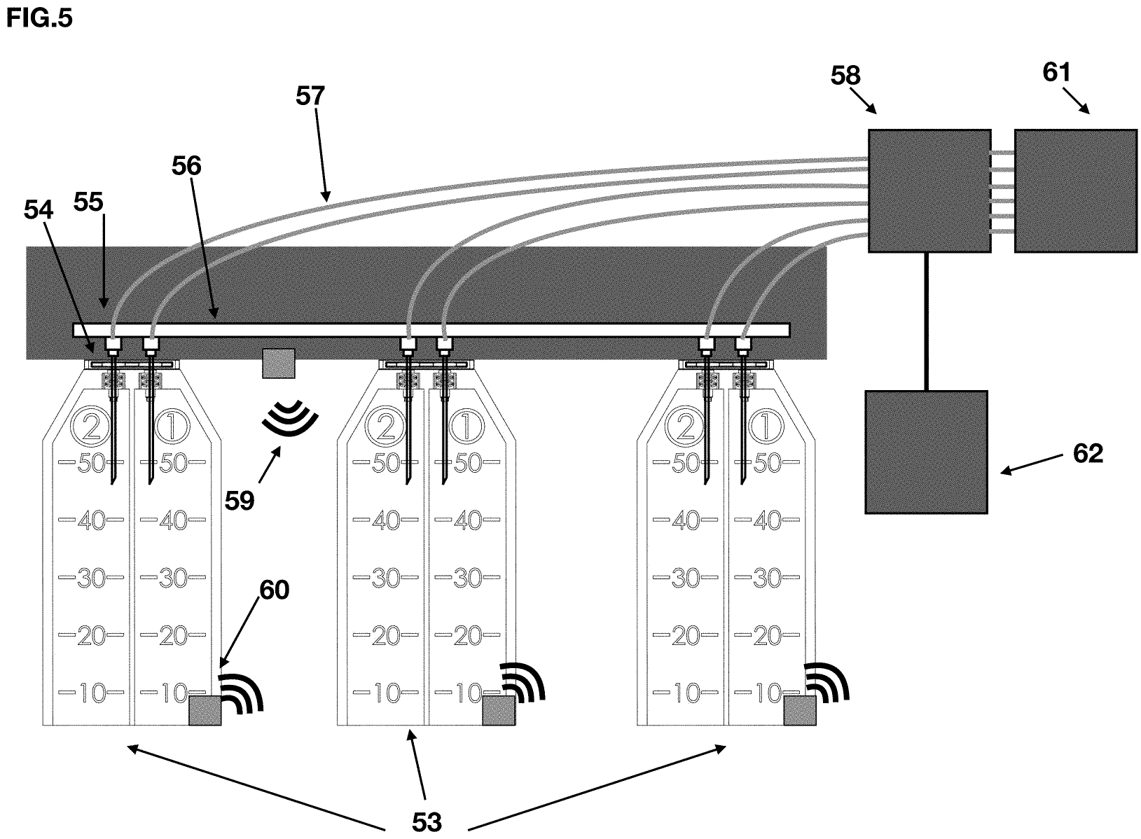

FIG. 5 shows one embodiment of a system for the analysis of the collected sample. In this embodiment, the sample collection units are connected to an automated system for the retrieval, processing and analysis, data storage, and residual sample storage, however one or all of these steps may be partially automated or fully manual. To allow for high-throughput of samples, several sample-collection units may be collected to the system concurrently via the same mechanism 54 used to connect the sample collection units to the device as described in the detailed description of FIG. 2A-2B. Stationary or automated mechanisms 55 may transfer the sample from the collection unit to one or more vessels or machines to be further processed. Prior to the removal of sample, the automated mechanism may centrifuge one or more of the samples concurrently or separately using the sample-collection apparatus as the sample-containment vehicle for centrifugation. In the case of an automated sample removal mechanism, a gantry or robotic shuttle mechanism 56 may facilitate the movement of the sample transfer mechanism from one sample-collection unit to another, or from sample-collection unit to some other vessel or machine to facilitate further processing or analysis. A processing or analysis mechanism 58 may be used to further process or analyze the sample for characteristics including, but not limited to those elucidated by fluid cytology, and turbidity, the presence and characteristics of rare cells such as circulating tumor cells, proteins such as the carcinoembryonic antigen (CAE) and MUC-1, chemokines, growth factors and cytokines, cellular debris including cytoplasmic fluid, cytosol, peptides, enzymes, and proteins, gene profile, pH, cell count, presence of blood, presence of bacteria or other pathogens or infectious material or evidence of such, cell surface receptors or other markers for relevant disease states or conditions. Individual fluid paths 57 may be employed to preserve the uniqueness of the samples. Sensors or readers 59 installed or incorporated in the mechanism may be used to read the RFID, barcode, QR code or other data-storage device 60 incorporated in the sample-collection mechanism. These may be one-way or two-way communication protocols. Data read or transferred may include, but are not limited to date of collection, elapsed time of collection, sample amount, sample chemical or biological characteristic, sample temperature at any or all times during collection, patient or sample identifying information, or shipping information. Once all processing is complete, the collected sample, in either processed or unprocessed form may be stored indefinitely by some means 61 for future processing, analysis, or other purposes. All data may likewise be stored indefinitely by some means 62 for future processing, analysis, or relay of pertinent information to the patient, caregiver, healthcare provider, or interested party. Machine learning, or large data-set processing algorithms may be used in the processing or analysis of the data.

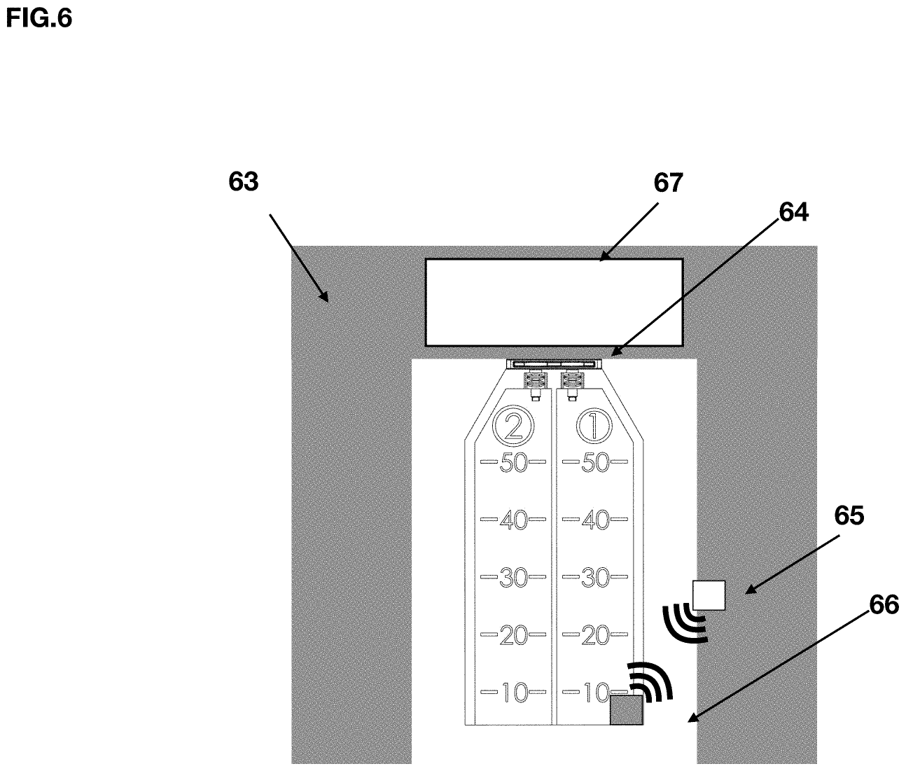

FIG. 6 shows on embodiment of a dock, or manual, decentralized version of the machinery described in the detailed description of FIG. 5. This embodiment may be placed in the home, clinic, hospital, pharmacy, or other inherently decentralized location for the processing, analysis, and storage of the sample and associated data, or for relay or immediate display of pertinent information to the patient, caregiver, healthcare provider, or interested party. The dock 63 may possess a means 64 for connecting to the sample-collection device using the same method described in the detailed description of FIG. 5. Sensors or readers 65 installed or incorporated in the mechanism may be used to read the RFID, barcode, QR code or other data-storage device 66 incorporated in the sample-collection mechanism. These may be one-way or two-way communication protocols. Data read or transferred may include, but are not limited to date of collection, elapsed time of collection, sample amount, sample chemical or biological characteristic, sample temperature at any or all times during collection, patient or sample identifying information, or shipping information. The dock may be used to further process or analyze the sample for characteristics. These characteristics include, but are not limited to those elucidated by fluid cytology, and turbidity, the presence and characteristics of rare cells such as circulating tumor cells, proteins such as the carcinoembryonic antigen (CAE) and MUC-1, chemokines, growth factors and cytokines, cellular debris including cytoplasmic fluid, cytosol, peptides, enzymes, and proteins, gene profile, pH, cell count, presence of blood, presence of bacteria or other pathogens or infectious material or evidence of such, cell surface receptors or other markers for relevant disease states or conditions. The dock may possess a cartridge or removable set of internal components to preserve the uniqueness of different samples from the same or different patients. The dock may possess a screen 67 to display results or clinically relevant information about the collected sample, or may transfer this information to the patient, caregiver, healthcare professional, or interested party.

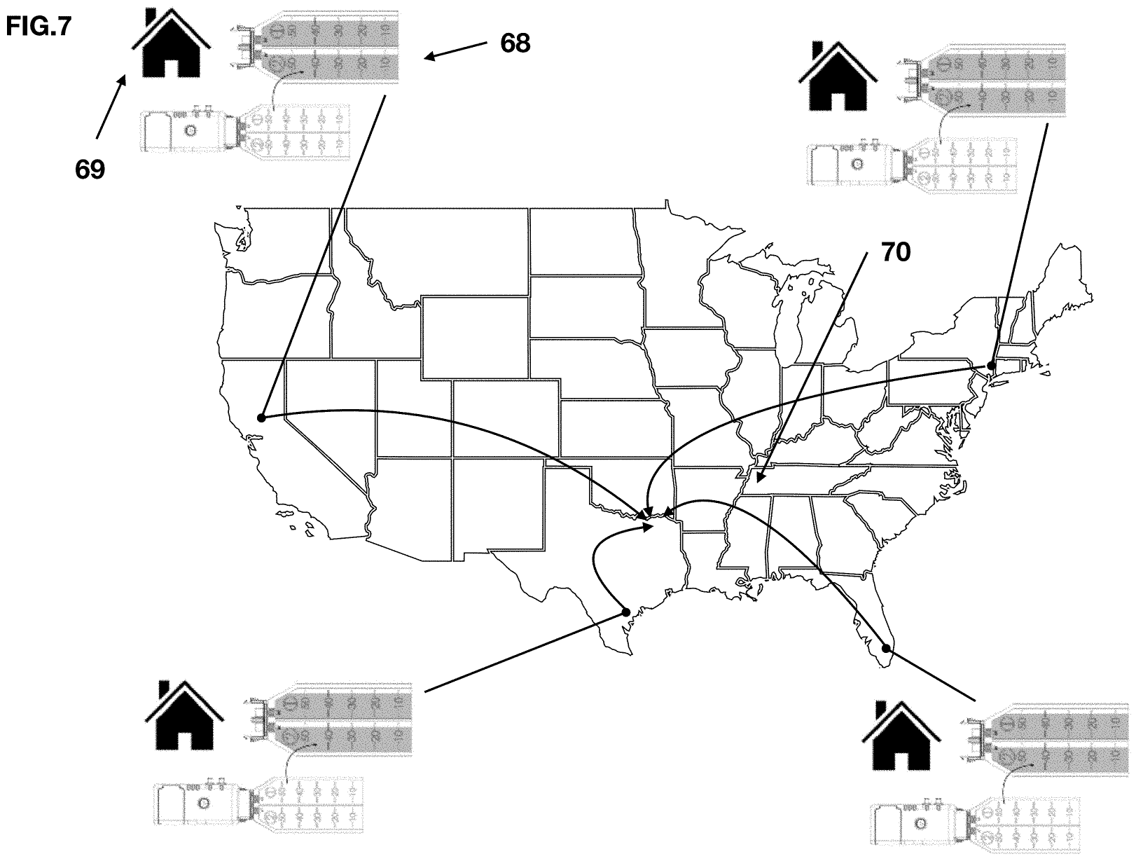

FIG. 7 shows one embodiment of a shipping scheme for the transfer of the sample-collection apparatus 68 from the home environment 69 to a centralized processing facility 70. The centralized processing facility may contain one or more of the machinery described in FIG. 5. Alternatively, all processing may be performed manually. The sample-collection apparatus and its associated packaging may be constructed to control, either actively or passively, characteristics of the sample and any associated packaging which include, but are not limited to the following: temperature, humidity, UV light transmittance and absorbance, shock and vibration, fluid ingress or egress, pH, gas ingress, egress, ambient concentration or absorbance, or any other biologically, clinically, or physically relevant characteristic.

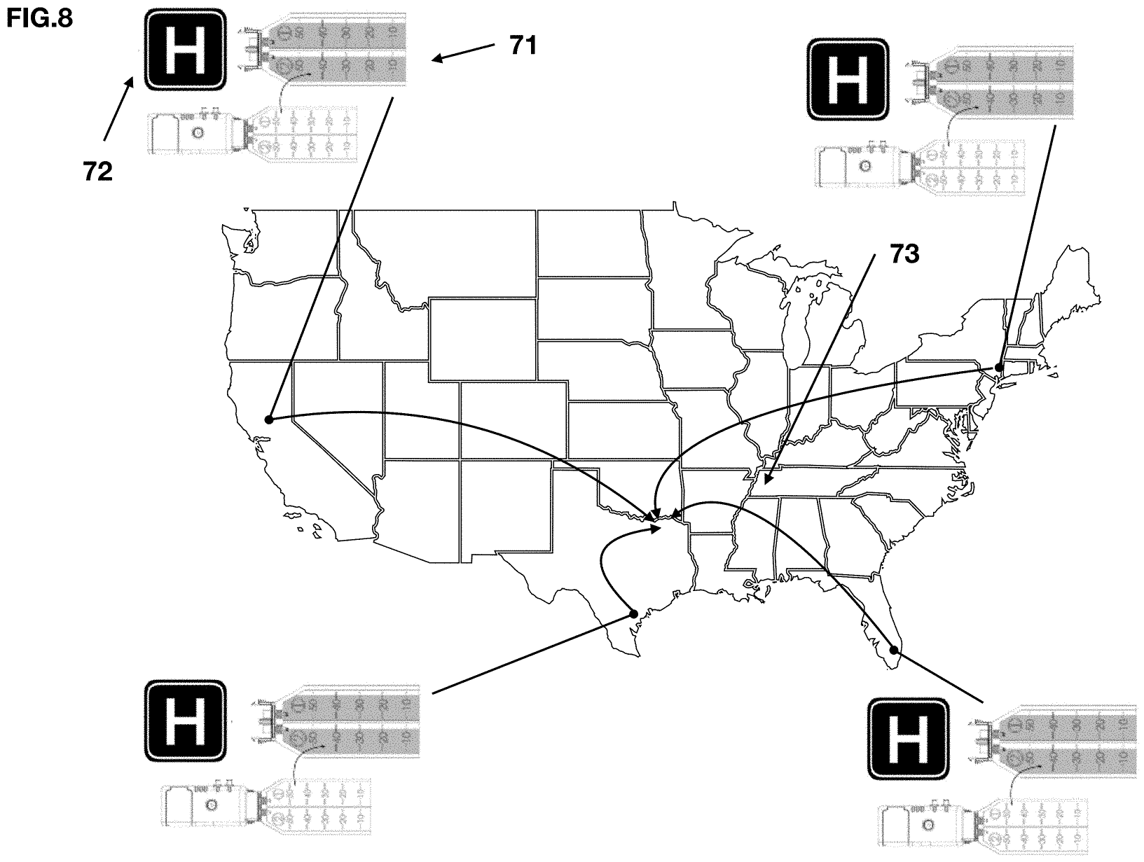

FIG. 8 shows one embodiment of a shipping scheme for the transfer of the sample-collection apparatus 71 from the hospital or clinic environment 72 to a centralized processing facility 73. The centralized processing facility may contain one or more of the machinery described in FIG. 5. Alternatively, all processing may be performed manually. The sample-collection apparatus and its associated packaging may be constructed to control, either actively or passively, characteristics of the sample and any associated packaging which include, but are not limited to the following: temperature, humidity, UV light transmittance and absorbance, shock and vibration, fluid ingress or egress, pH, gas ingress, egress, ambient concentration or absorbance, or any other biologically, clinically, or physically relevant characteristic.

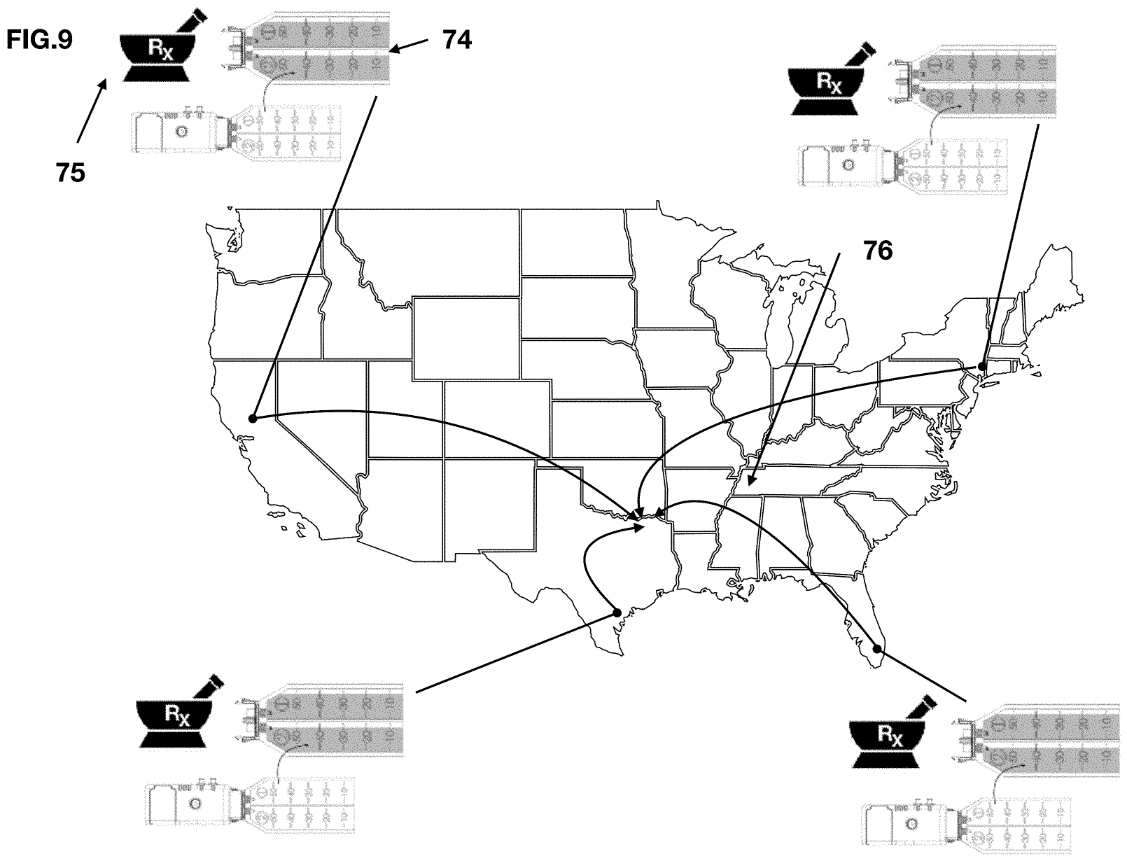

FIG. 9 shows one embodiment of a shipping scheme for the transfer of the sample-collection apparatus 74 from the pharmacy environment 75 to a centralized processing facility 76. The centralized processing facility may contain one or more of the machinery described in FIG. 5. Alternatively, all processing may be performed manually. In this embodiment, the patient may bring themselves, or transfer by courier to the pharmacy, the sample-collection unit to facilitate easy shipping. The sample-collection apparatus and its associated packaging may be constructed to control, either actively or passively, characteristics of the sample and any associated packaging which include, but are not limited to the following: temperature, humidity, UV light transmittance and absorbance, shock and vibration, fluid ingress or egress, pH, gas ingress, egress, ambient concentration or absorbance, or any other biologically, clinically, or physically relevant characteristic.

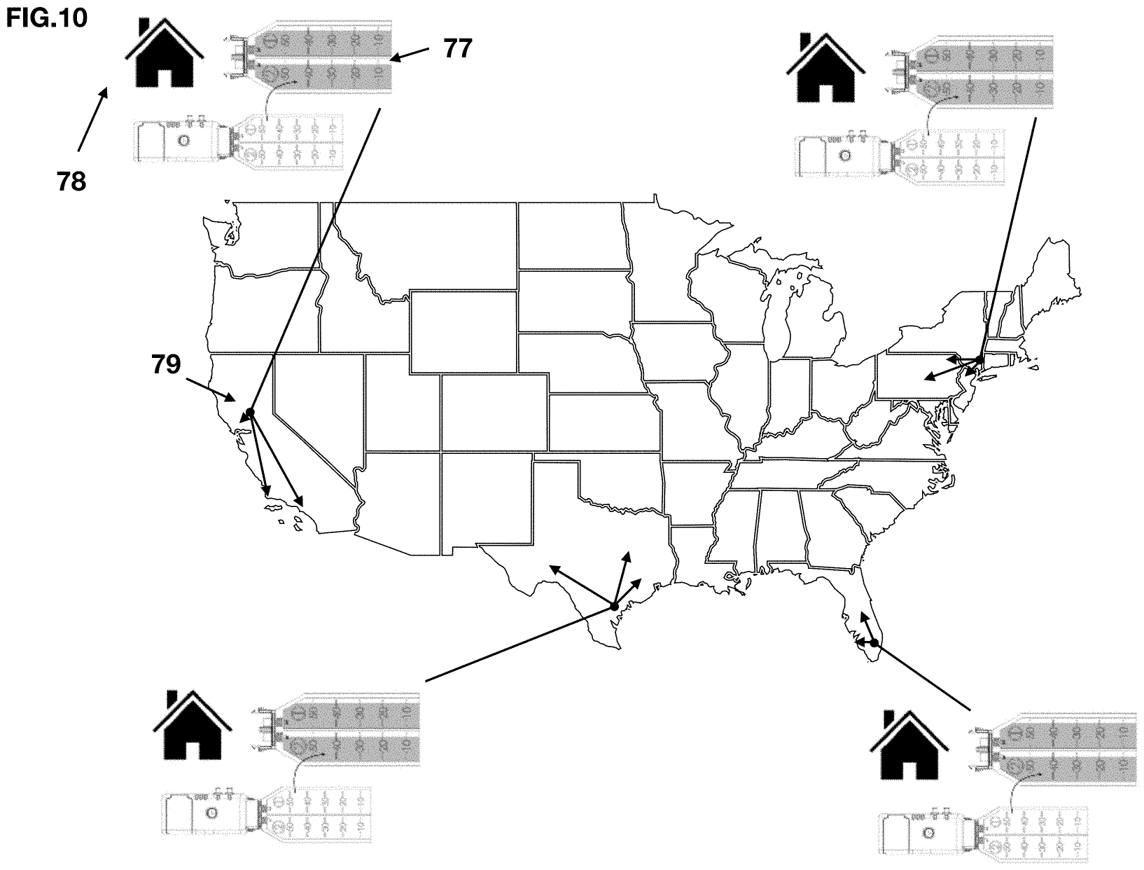

FIG. 10 shows one embodiment of a shipping scheme for the transfer of the sample-collection apparatus 77 from the home environment 78 to a decentralized processing facility 79. The centralized processing facility may contain one or more of the machinery described in FIG. 5. Alternatively, all processing may be performed manually. The sample-collection apparatus and its associated packaging may be constructed to control, either actively or passively, characteristics of the sample and any associated packaging which include, but are not limited to the following: temperature, humidity, UV light transmittance and absorbance, shock and vibration, fluid ingress or egress, pH, gas ingress, egress, ambient concentration or absorbance, or any other biologically, clinically, or physically relevant characteristic.

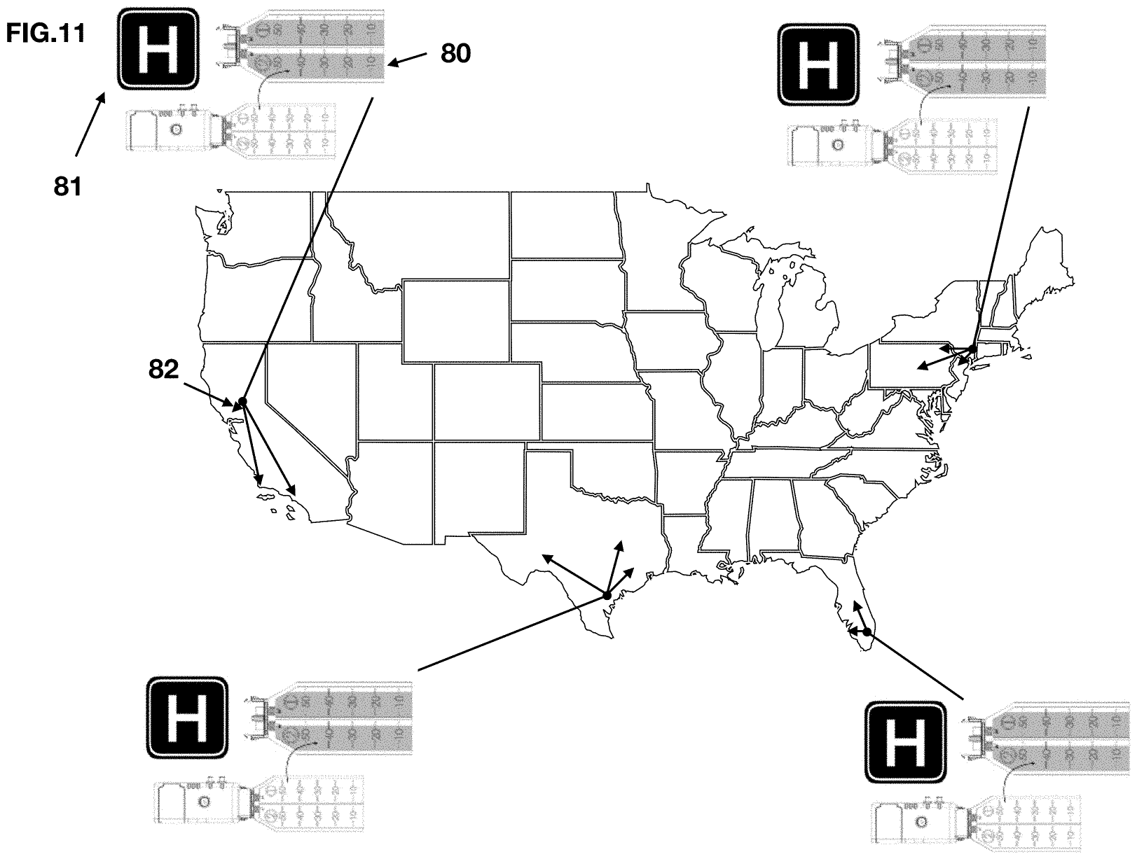

FIG. 11 shows one embodiment of a shipping scheme for the transfer of the sample-collection apparatus 80 from the hospital or clinic environment 81 to a decentralized processing facility 82. The centralized processing facility may contain one or more of the machinery described in FIG. 5. Alternatively, all processing may be performed manually. The sample-collection apparatus and its associated packaging may be constructed to control, either actively or passively, characteristics of the sample and any associated packaging which include, but are not limited to the following: temperature, humidity, UV light transmittance and absorbance, shock and vibration, fluid ingress or egress, pH, gas ingress, egress, ambient concentration or absorbance, or any other biologically, clinically, or physically relevant characteristic.

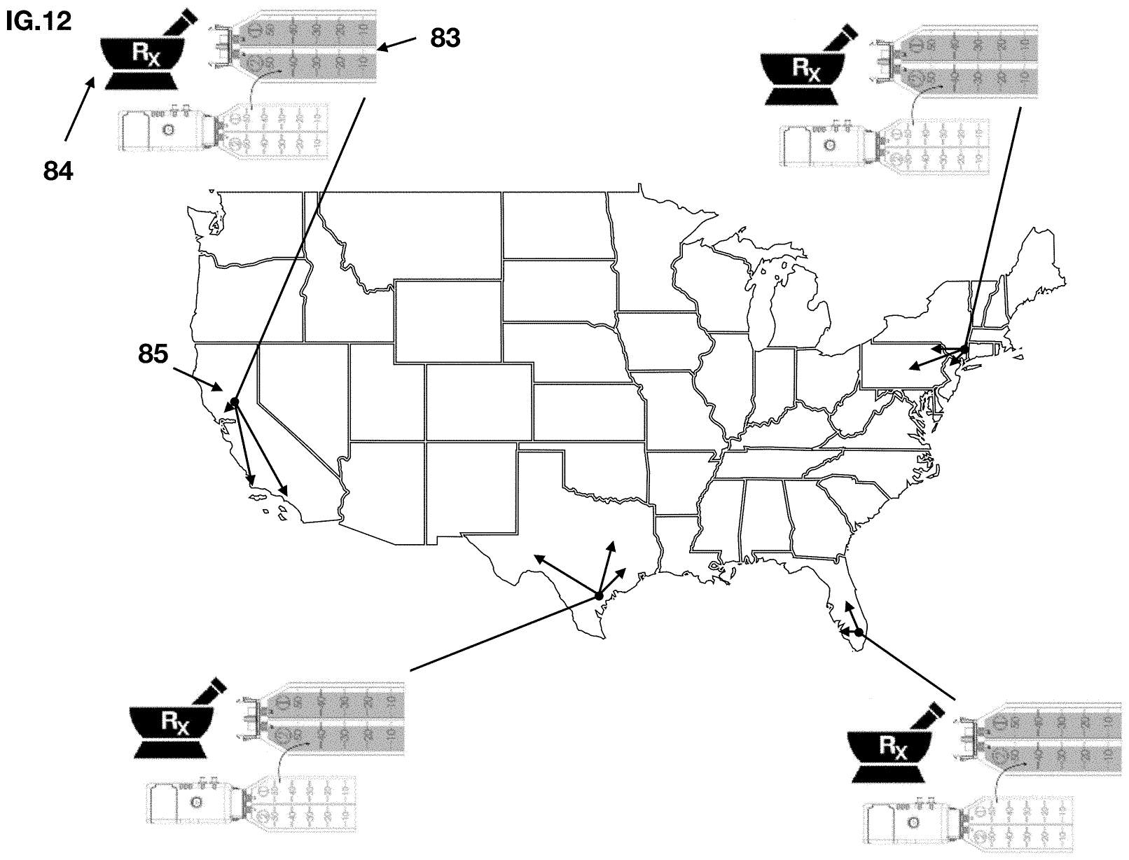

FIG. 12 shows one embodiment of a shipping scheme for the transfer of the sample-collection apparatus 83 from the pharmacy environment 84 to a decentralized processing facility 85. The centralized processing facility may contain one or more of the machinery described in FIG. 5. Alternatively, all processing may be performed manually. In this embodiment, the patient may bring themselves, or transfer by courier to the pharmacy, the sample-collection unit to facilitate easy shipping. The sample-collection apparatus and its associated packaging may be constructed to control, either actively or passively, characteristics of the sample and any associated packaging which include, but are not limited to the following: temperature, humidity, UV light transmittance and absorbance, shock and vibration, fluid ingress or egress, pH, gas ingress, egress, ambient concentration or absorbance, or any other biologically, clinically, or physically relevant characteristic.

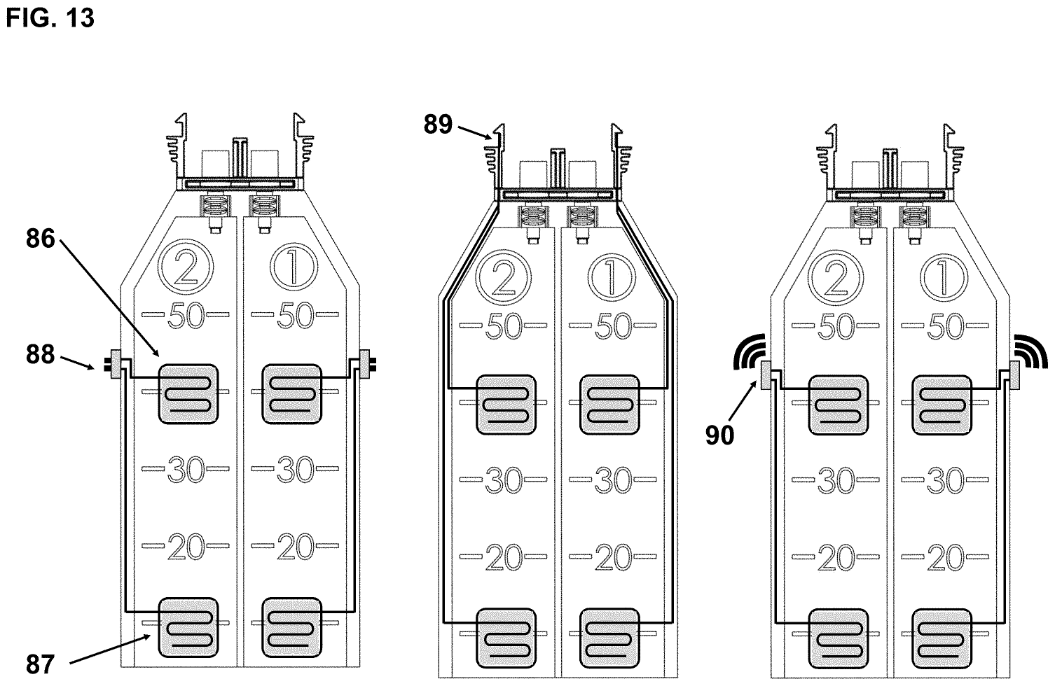

FIG. 13 shows one embodiment of the sample collection unit which possess at least one sensor internal to the collection unit, which may be used analyze to at least one chemical or biochemical, physical or biophysical, optical, electromagnetic, or other relevant characteristic of the fluid, constituents of the fluid, or any byproducts which may form in the sample collection unit either naturally, or as a result of intentional modification of the sample or sample environment. In this embodiment, the sensor 86 may be used in conjunction with one or more additional sensors 87 to analyze the same or multiple properties of the fluid, either in conjunction with one another or separately. In this embodiment, a port 88 for making connection, electrical or otherwise, with an external device for measurement, for signal processing, or for data storage, communication, or transmission with a physician, patient, or other interested party by cellular, near-field communication (NFC), Bluetooth, wi-fi, or other analog or digital communication method is placed on the exterior of the collection unit. In another embodiment, the port 89 is placed on the collection unit in a manner which facilitates connection to the suction apparatus. Other embodiments of the collection unit may possess onboard devices 90 for wireless (e.g. cellular, near-field communication (NFC), Bluetooth, wi-fi, etc.) communication with physician, patient, other interested party, or with an external device for measurement, for signal processing, or for data storage, communication, or transmission with a physician, patient, or other interested party by cellular, near-field technology, Bluetooth, wi-fi, or other analog or digital communication method

Thus, a manner of providing a means of uniformly collecting fluids and related materials from a patient (whether from a surgical wound, externally caused wound, or internal fluid/material buildup) for analysis thereof in a controlled manner is permitted. Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments, and it is intended that the description herein cover any and all such applications, modifications, and embodiments within the scope of the present invention.

What we claim is:

1. A system for analyzing collected bodily fluids and related materials from a live patient, said system including at least one vacuum device attached to at least one fluid removal line, at least one collection component associated with both of said at least one vacuum device and said at least one fluid removal line, and at least one medical diagnostic instrument; wherein said at least one fluid removal line has an open end distal to said at least one vacuum device and configured for placement within or on a surgical wound; wherein said at least one vacuum device operates to transfer fluid from said wound to said at least collection component through said at least one fluid removal line; wherein said at least one collection component provides said collected surgical and/or wound fluid for introduction within said at least one medical diagnostic instrument; and wherein said at least one vacuum device provides a controlled rate of surgical and/or fluid removal from said live patient at specified times.

2. The system of claim 1 wherein said at least one collection component includes fluid analysis capabilities or enables/facilitates fluid analysis pertaining to at least one chemical or biochemical, physical or biophysical, optical, electromagnetic, or other relevant characteristic of the fluid, constituents of the fluid, or any byproducts which may form in the sample collection unit either naturally, or as a result of intentional modification of the sample or sample environment .

3. The system of claim 1 wherein said at least one collection component includes equipment or materials intended to control, either actively or passively, characteristics of the sample or sample constituents which include, but are not limited to the following: temperature, humidity, UV light transmittance and absorbance, shock and vibration, fluid ingress or egress, pH, gas ingress, egress, ambient concentration or absorbance of constituents of the sample, or any other biologically, clinically, or physically relevant characteristic.

4. A method of collecting bodily fluids and related materials from a live patient, said method including: a) the provision of at least one vacuum device attached to at least one fluid removal line and leading to at least one collection component associated with both of said at least one vacuum device and said at least one fluid removal line; b) placing said at least one fluid removal line on or within the body of said live patient (e.g. surgical wounds, surface or closed wound, thoracic space, and pleural space) c) activating said at least one vacuum device at a controlled rate of fluid removal, thereby transferring surgical and/or wound fluid from said live patient to said at least one collection component; and d) discontinuing said collection component after a set period of time, leaving a resultant collection of removed fluid from said live patient; wherein said at least one collection component includes fluid analysis capabilities or enables/facilitates fluid analysis pertaining to at least one chemical or biochemical, physical or biophysical, optical, electromagnetic, or other relevant characteristic of the fluid or the constituents of the fluid.

5. The method of claim 3 wherein said collected fluid is further transferred to at least one medical diagnostic instrument for analysis.

6. The method of claim 3 wherein said collected fluid is evaluated, characterized, or analyzed while still in the sample collection unit by equipment or analysis modalities external to the sample collection unit.

7. The method of claim 3 wherein said collected fluid is evaluated, characterized, or analyzed while still in the sample collection unit by equipment or analysis modalities internal to, or onboard the sample collection unit.

8. The method of claim 3 wherein said at least one collection component includes equipment or materials intended to control, either actively or passively, characteristics of the sample or sample constituents which include, but are not limited to the following: temperature, humidity, UV light transmittance and absorbance, shock and vibration, fluid ingress or egress, pH, gas ingress, egress, ambient concentration or absorbance of constituents of the sample, or any other biologically, clinically, or physically relevant characteristic.
